(19) United States
(12) Patent Application Publication
(10) Pub. No.: US 2021/0251903 A1
(43) Pub. Date: Aug. 19, 2021

(54) Preparation of Microparticulate Triamcinolone Acetonide Injectable Suspension

(71) Applicant: Somerset Therapeutics LLC, Hollywood, FL (US)

(72) Inventors: Rahul Kalhapure, Somerset, NJ (US); Prem Sagar Akasapu, Somerset, NJ (US); Veerappan Subramanian, Somerset, NJ (US); Ilango Subramanian, Somerset, NJ (US)

(73) Assignee: Somerset Therapeutics LLC, Hollywood, FL (US)

(21) Appl. No.: 16/791,825

(22) Filed: Feb. 14, 2020

(51) Int. Cl. A61K 9/16; A61K 9/107; A61K 31/58; A61K 9/00

(52) U.S. Cl. CPC A61K 9/1694; A61K 9/1617; A61K 9/1611; A61K 9/0019; A61K 9/107; A61K 31/58; A61K 9/1652

(57) ABSTRACT

The present invention relates to a process for preparation of a water-insoluble steroid composition by moist heat sterilization. Particularly, the invention relates to a process for preparation of a water-insoluble steroid composition comprising moist heat sterilization of an aqueous slurry of the steroid triamcinolone acetonide in the presence of benzyl alcohol, sodium chloride and sodium carboxymethylcellulose under a nitrogen atmosphere. The suspensions prepared by using the current invention exhibited good physical and chemical stability. Compositions related thereto are also disclosed.

### FIELD OF THE INVENTION

Disclosed herein are processes for the preparation of a water-insoluble steroid suspension, particularly a process for making a triamcinolone acetonide suspension that includes moist heat sterilization. The present invention also relates to water-insoluble steroid suspension compositions suitable for parenteral administration.

### BACKGROUND OF THE INVENTION

Triamcinolone acetonide is a synthetic glucocorticoid corticosteroid with anti-inflammatory action. Triamcinolone acetonide is 9-Fluoro-11β, 16α, 17, 21-tetrahydroxypregna-1, 4-diene-3, 20-dione cyclic 16, 17-acetal with acetone. Its structural formula is:

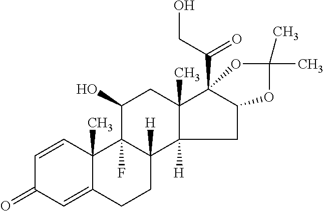

Triamcinolone acetonide suspension for injection is approved as 10 mg/ml, 40 mg/ml and 80 mg/ml strengths under the brand name Kenalog® by Apothecon with routes of administration of intra-muscular or intra-articular use. Triamcinolone acetonide administered intra-muscularly is used for allergy, dermatologic, endocrine, gastrointestinal, hematologic, neoplastic, nervous, ophthalmic, renal, respiratory, rheumatic and other miscellaneous disease or disorders. Triamcinolone acetonide suspension administered intra-articularly is used for adjunctive therapy for short-term administration in acute gouty arthritis; acute rheumatic carditis; ankylosing spondylitis; psoriatic arthritis; rheumatoid arthritis, including juvenile rheumatoid arthritis (selected cases may require low-dose maintenance therapy), dermatomyositis, polymyositis, and systemic lupus erythematosus.

A pharmaceutical suspension is a coarse dispersion in which insoluble solid particles are dispersed in a liquid medium. Suspensions contribute to pharmacy and medicine by supplying insoluble and often distasteful substances in a form that is pleasant to the taste, by providing a suitable form for the application of dermatological materials to the skin and sometimes to the mucous membranes, and for the parenteral administration of insoluble drugs. Therefore pharmaceutical suspensions may be classified into three groups: orally administered mixtures, externally applied lotions and injectable preparations.

Injectable suspension formulations are administered by intravenous (IV), subcutaneous (SC) or intramuscular (IM) routes. Parenteral suspensions are heterogeneous systems that typically consist of a solid phase dispersed in a liquid phase, the liquid phase being aqueous or nonaqueous. To be effective and pharmaceutically acceptable, injectable suspensions should preferably be sterile, stable, resuspendable, syringeable, injectable, isotonic and nonirritating.

A pharmaceutical suspension preparation possesses certain desirable qualities, including the following: i) the suspended material should not settle rapidly; ii) the particles that do settle to the bottom of the container must not form a hard cake but should be readily re-dispersed into a uniform mixture when the container is shaken; and iii) the suspension must not be too viscous to pour freely from the orifice of the bottle or to flow through a syringe needle.

It is important that the characteristics of the dispersed phase are chosen with care so as to produce a suspension having optimum physical, chemical and pharmacological properties. Particle size distribution, specific surface area, inhibition of crystal growth, and changes in the polymorphic form are of special significance and the formulator must ensure that these and other properties do not change sufficiently during storage to adversely affect the performance of the suspensions with aging.

The foregoing characteristics result in manufacturing, storage, and usage requirements that make injectable suspensions one of the most difficult dosage forms to develop.

In the field of injectable preparations, aqueous suspensions for parenteral administration have already been described in scientific and patent literature. These suspensions have been known for a long time and are routinely heat sterilized as ultrafiltration sterilization is not a good option, however, autoclave sterilization could result in degradation of the drug.

U.S. Pat. No. 3,962,430 A discloses an autoclaving method for sterilization of active pharmaceutical ingredients in the presence of sodium chloride. This method has been reported to eliminate the problem of change in particle size of dexamethasone acetate, lidocaine hydrochloride, hydrocortisone alcohol, prednisolone tertiary butyl acetate, indomethacin, thiabendazole, testosterone and estradiol.

U.S. Pat. No. 6,495,534 B2 discloses the preparation of stable aqueous suspensions of medroxyprogesterone acetate in a method that utilizes steam sterilization of an aqueous solution of excipients, followed by dispersion of the drug, followed by further homogenization and aseptic filling into the final containers.

U.S. Pat. No. 7,892,483 B2 discloses a process for the sterilization of a steroid that involves heat treating the steroid in the form of a wet mass consisting essentially of the steroid, water and surfactant.

PCT Application Number WO 1999/061001A1 discloses compositions of submicron to micron sized particles of water-insoluble biologically active substances that are stabilized by thermoprotecting agents, and can be terminally steam sterilized without any significant increase of mean particle size.

U.S. Patent Publication Number 2006/0094700 A1 discloses the effect of autoclaving (121° C. for 45 min) a 20% aqueous suspension of triamcinolone acetonide in the presence and absence of sodium chloride and buffer salts. The '700 patent publication discloses that the results of the examples shows that phosphate salts decrease the particle size, which the inventors state may be due to the breakup of some crystal aggregates, which is important for injectable suspensions where larger particles sediment rapidly and can contribute to blockage of the fine gauge needle.

U.S. Patent Publication Number 2019/0269616 A1 discloses terminal sterilization of a triamcinolone acetonide parenteral suspension by autoclaving at 121° C. for 15 minutes, 30 minutes, 45 minutes and 60 minutes. The patent publication also discloses that there was no change in triamcinolone acetonide assay value and impurities were not more than 2%, however, no information has been provided on the effect of autoclaving exposure on the particle size of the triamcinolone acetonide.

Thus, there exists an enduring need to develop a robust method for sterilizing parenteral suspension formulations having one or more water insoluble components, and which will provide an alternative to existing formulations. The inventors of the present invention have developed a process for sterilizing a water-insoluble steroid suspension comprising moist heat sterilization of an aqueous slurry of triamcinolone acetonide in the presence of benzyl alcohol, sodium chloride and sodium carboxymethylcellulose. The suspensions prepared by using the current invention exhibited good physical and chemical stability.

### SUMMARY OF THE INVENTION

The present invention provides a process for preparation of sterilized injectable suspensions by moist heat sterilization. Particularly, the invention relates to a process for sterilizing a water-insoluble steroid suspension comprising moist heat sterilization of an aqueous slurry of water-insoluble steroid in the presence or absence of benzyl alcohol, sodium chloride and sodium carboxymethylcellulose.

In another general aspect, there is provided a process for preparation of a sterilized injectable water-insoluble steroid suspension by moist heat sterilization.

In another general aspect, there is provided a process for sterilizing a water-insoluble steroid suspension comprising moist heat sterilization of an aqueous slurry of water-insoluble steroid in the presence of benzyl alcohol, sodium chloride and sodium carboxymethylcellulose.

In another general aspect, there is provided a process for sterilizing a water-insoluble steroid suspension comprising moist heat sterilization of an aqueous slurry of triamcinolone acetonide in the presence of benzyl alcohol, sodium chloride and sodium carboxymethylcellulose.

In another general aspect, there is provided a process for sterilizing triamcinolone acetonide suspension comprising moist heat sterilization of an aqueous slurry of triamcinolone acetonide in the presence of benzyl alcohol, sodium chloride and sodium carboxymethylcellulose under nitrogen atmosphere.

In one general aspect, there is provided a process for preparing a sterilized injectable triamcinolone acetonide suspension, wherein the particle size of the triamcinolone acetonide particles in the suspension ranges from about 0.5 μm to about 30 μm.

In one general aspect, there is provided a process for preparing a sterilized injectable triamcinolone acetonide suspension, wherein the particle size of triamcinolone acetonide in the suspension increased compared to the particle size in the slurry, and ranges from about 0.5 μm to about 30 μm. In one general aspect, there is provided a process for preparing a sterilized injectable triamcinolone acetonide suspension, wherein the viscosity of the suspension can be controlled in the range of 8-50 centipoise.

In one general aspect, there is provided a process for preparing a sterilized injectable triamcinolone acetonide suspension, wherein the concentration of triamcinolone acetonide in a slurry used to make the suspension can vary from about 1% to about 20%.

In one general aspect, there is provided a process for preparing a sterilized injectable triamcinolone acetonide suspension, wherein the dissolution profile of the suspension is identical to the reference product.

In one general aspect, there is provided a process for preparing a sterilized injectable triamcinolone acetonide suspension, wherein the suspension has a Difference Factor (F₁) less than 9 and the Similarity Factor (F₂) values greater than 60 as compared to the reference product.

In one general aspect, there is provided a sterilized injectable suspension composition comprising a water insoluble steroid drug and pharmaceutically acceptable excipients.

In one general aspect, there is provided a sterilized injectable suspension composition comprising triamcinolone acetonide and one or more pharmaceutically acceptable excipients selected from the group consisting of suspending agents, viscosity enhancers, preservatives, surfactants, buffering agents and vehicle.

In one general aspect, there is provided a sterilized injectable suspension comprising about 1% to about 15% triamcinolone acetonide, about 0.66% sodium chloride, about 0.04% polysorbate 80, about 0.99% benzyl alcohol and about 0.63% carboxymethylcellulose sodium.

In one general aspect, there is provided a sterilized injectable suspension comprising about 1.96% to about 19.6 triamcinolone acetonide, about 1.65% to about 1.94% benzyl alcohol, about 1.1% to about 1.29% sodium chloride, about 1.05% to about 1.23% sodium carboxymethylcellulose and one or more other pharmaceutically acceptable excipients.

In one general aspect, there is provided a process for preparation of a sterilized injectable suspension composition comprising triamcinolone acetonide and a unit package formulation of the same.

In one general aspect, there is provided a process for preparation of a sterilized injectable suspension composition comprising triamcinolone acetonide and one or more pharmaceutically acceptable excipients comprising the following steps:

a) In a suitable container dissolve benzyl alcohol, sodium chloride, and carboxymethylcellulose sodium in water for injection and transfer to the second container of step (b),
b) In the second container, add the required quantity of triamcinolone acetonide to from a slurry, homogenize, filter and sterilize at 122° C. for about 15 to about 90 minutes under nitrogen atmosphere,
c) In a third suitable container, dissolve polysorbate 80 in water for injection, filter and transfer aseptically to the suspension of step (b),
d) Transfer the obtained suspension of step (c) to a sterile holding container for filing into vials of the required volume.

In another general aspect, there is provided a process for preparing a sterilized injectable triamcinolone acetonide suspension. The suspension is characterized in that the dosage form retains at least 90% w/w of the potency of triamcinolone acetonide when stored at 25° C. and 60% relative humidity or at 40° C. and 75% relative humidity for 3 months.

In another general aspect, there is provided a process for preparing a sterilized injectable triamcinolone acetonide suspension, wherein the obtained suspension is aseptically distributed into single dose or multidose containers.

In another general aspect, there is provided a process for preparing a sterilized injectable triamcinolone acetonide suspension, wherein the obtained suspension can be easily resuspended and can easily flow through a syringe needle for intradermal, subcutaneous and intramuscular administration.

In another general aspect, there is provided a process for preparing a sterilized injectable triamcinolone acetonide suspension, wherein the obtained suspension exhibits good stability throughout the shelf life as the impurities observed are well below the specified limits.

### BRIEF DESCRIPTION OF THE DRAWING FIGURES

FIG. 1 is a flow chart representation of the manufacturing process for a Triamcinolone Acetonide injectable suspension.

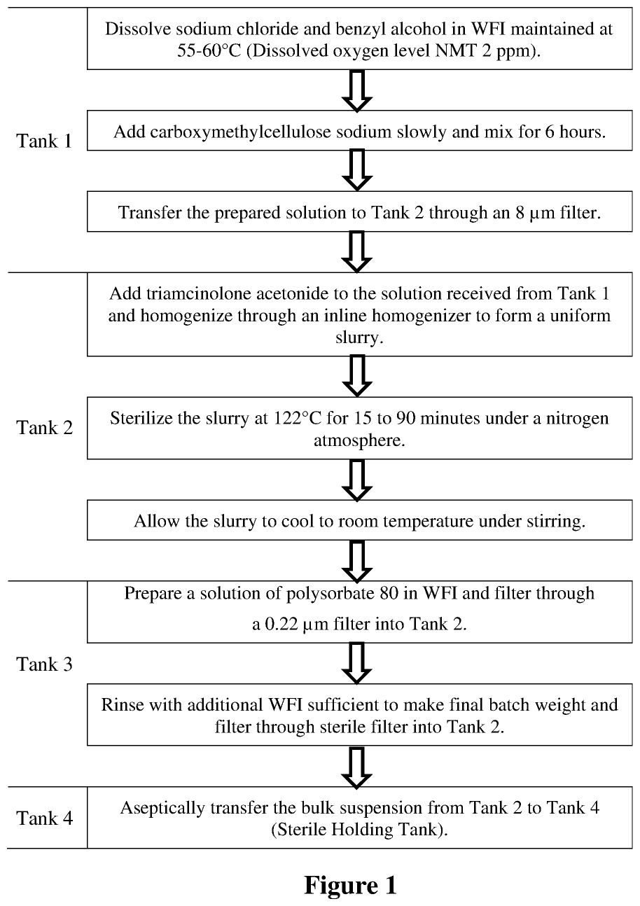

FIG. 2 is a dissolution profile comparison of different formulations of triamcinolone acetonide injectable suspension, USP (40 mg/mL) with the Reference Product (RLD).

### DETAILED DESCRIPTION OF THE INVENTION

The present invention provides a process for preparation of a sterilized injectable suspension by moist heat sterilization. In particular, the invention relates to a process for sterilizing a water-insoluble steroid composition comprising moist heat sterilization of an aqueous slurry of a water-insoluble steroid in the presence of benzyl alcohol, sodium chloride and sodium carboxymethylcellulose.

The term “water-insoluble steroid” is a steroid which is not completely dissolved at the concentration at which it is administered in an aqueous composition. Thus, depending upon the use and concentration, a steroid may be considered water-insoluble in one situation but not water-insoluble in another situation. While not intending to limit the scope of the invention in any way, the water-insoluble steroid referred to in the present invention relates to the pharmaceutically active agents or their pharmaceutically acceptable salts suitable for parenteral use and/or normally supplied as suspension formulation. Such active agents include triamcinolone acetonide, methyl prednisolone, cortisone, cortisone acetate, dexamethasone, dexamethasone acetate, dexamethasone tertiary butyl acetate, hydrocortisone acetate, prednisolone acetate, betamethasone acetate, betamethasone, fluticasone propionate, budesonide, tipredane, dexamethasone, beclomethasone diproprionate, prednisolone, flucinolone, mometasone furoate, rofleponide palmitate, flumethasone, flunisolide, ciclesonide, deflazacort and cortivazol.

Often steroids are administered as ester, acetal, or ketal prodrugs, many of which are water-insoluble. These prodrugs are also considered to be steroids.

A “prodrug”, as generally understood in the art, is a compound which is converted to a therapeutically active compound in vivo after administration, and the term should be interpreted as broadly herein as is generally understood in the art. While not intending to limit the scope of the invention, conversion may occur by hydrolysis of an ester group or some other biologically labile group.

While not intending to limit the scope of the invention in any way, triamcinolone actetonide is the preferred water-insoluble steroid suspension prepared according to the present invention.

The amount of triamcinolone actetonide used in the suspension is about 1% to about 10% by weight.

The compositions include water insoluble steroid suspensions suitable for parenteral administration and one or more pharmaceutically acceptable excipients selected from the group consisting of suspending agents, viscosity enhancers or thickening agents, preservatives, surfactants, buffering agents and vehicle.

Exemplary surfactants include, but are not limited to, polyoxyethylene (20) sorbitan monooleate (polysorbate 80), polyoxyethylene (20) sorbitan monolaurate (polysorbate 20), polyoxyethylene (20) sorbitan monostearate (polysorbate 60), polyoxyl 40 stearate, polyoxyethylene 50 stearate, sodium lauryl sulfate, and the like. The term surfactants as used herein is interchangeable with wetting agents.

Suspending agents are needed in suspension compositions to provide a suspension in which the settling of the particles is impeded and at the same time remains sufficiently fluid to be syringeable. Exemplary suspending agents include, but are not limited to, sodium carboxymethylcellulose, povidone, polyvinylpyrrolidone compounds and polyethylene glycols. Preferred examples of polyethylene glycols are those having a molecular weight from about 300 to about 6000, e.g. polyethylene glycol 3350 and polyethylene glycol 4000. Preferred polyvinylpyrrolidone compounds according to the invention are those having a molecular weight from about 7000 to about 54000, for instance PVP K12, K17, K25 and K30, in particular K12 and K17, PVP K17 being the most preferred. Other suitable suspending agents include thickening or viscosity agents such as, for instance, well known cellulose derivatives for example methylcellulose, carboxymethylcellulose, hydroxyethylcellulose and hydroxypropylmethylcellulose), gelatin and acacia, in particular methylcellulose.

Thickening agents are used in the suspension compositions to improve the form of the formulation for convenient administration and to improve bioavailability. Exemplary thickening agents include, but are not limited to, polymers containing hydrophilic groups such as monosaccharides and polysaccharides, ethylene oxide groups, hydroxyl groups, carboxylic acids or other charged functional groups. While not intending to limit the scope of the invention, some examples of useful viscosity-enhancing agents are sodium carboxymethylcellulose, hydroxypropylmethylcellulose, povidone, polyvinyl alcohol, and polyethylene glycol.

The tonicity agents are used in suspension compositions to adjust the composition of the formulation to be within the desired isotonic range. Exemplary tonicity agents include, but are not limited to glycerin, mannitol, sorbitol, sodium chloride, and other electrolytes.

The buffering agents or pH-adjusting agent are used in suspension compositions to adjust the pH to a desirable range. Exemplary buffers are well known by those skilled in the art and include acetate, borate, carbonate, citrate, histidine, and phosphate buffers. While not intending to limit the scope of the invention in any way, certain compositions disclosed herein have a pH of from 4 to 8.

The preservatives in the suspension compositions are used to inhibit the microbial growth. Suitable preservatives include, but are not limited to, hydrogen peroxide; sorbic acid; biquanides; quaternary ammonium salts such as benzalkonium chloride and benzethonium chloride; cationic compounds such as chlorhexidine gluconate; p-hydroxybenzoates such as methyl p-hydroxybenzoate, ethyl p-hydroxybenzoate, propyl p-hydroxybenzoate and butyl p-hydroxybenzoate; alcohol compounds such as chlorobutanol and benzyl alcohol; sodium dehydroacetate; thiomersal and the like.

Heating is carried out for the length of time required to kill the pathogens of importance to the application in which the composition is used. Such a determination is well within the skill of a person of ordinary skill in the art. The heat sterilization of the slurry may be performed at a temperature of about 122° C., or over a range of about 115° C. to about 128° C. For example, in the compositions herein, the heating is maintained at or near the peak temperature for at least about 15 to about 90 minutes.

The sterilized injectable suspension may include about 1.96% to about 19.6% triamcinolone acetonide, about 1.65% to about 1.94% benzyl alcohol, about 1.1% to about 1.29% sodium chloride, about 1.05% to about 1.23% sodium carboxymethylcellulose and one or more other pharmaceutically acceptable excipients.

The particle size of triamcinolone acetonide in the suspension prepared according to the present invention ranges from about 0.5 μm to about 30 μm. It should be understood that the particle size of the triamcinolone acetonide in the suspension is greater than the particle size of the triamcinolone acetonide in the slurry because the moist heat sterilization increases the particle size of the active ingredient particles.

The dissolution profile of the triamcinolone acetonide suspension prepared according to the present invention is identical to the reference product.

The dissolution profile of triamcinolone acetonide suspension prepared according to the present invention is compared with reference products/marketed products using two factors; Difference Factor (F₁) and Similarity Factor (F₂).

f1={[Σt=1n|R−T|]/[Σt=1nR]}×100

f₂=50 log {[1+(1/n)Σₜ₌₁ⁿ(Rₜ−Tₜ)²]_{-0.5}*100}

In the Similarity Factor equation, Rt and Tt are the cumulative percentage dissolved at each of the selected n time points of the reference and test products, respectively. The Difference Factor (F₁) is used to calculate the percent difference between the two curves at each time point and is a measurement of the relative error between the two curves. The Similarity Factor (F₂) measures the closeness between the two profiles.

The Difference Factor (F₁) and Similarity Factor (F₂) are usually considered satisfactory if in the range of 0-15 and 50-100, respectively. The Difference Factor (F₁) and the Similarity Factor (F₂) values are reported in Table 1.

TABLE 1

| Reference Product | Test Product | F2 | F1 |
|---|---|---|---|
| ABF3407 | P006-131-05 | 78 | 4 |
| ABF3407 | P006-138-01 | 66 | 6 |
| ABF3407 | P025-08-02 | 63 | 8 |
| AAX1117 | P006-131-05 | 81 | 3 |
| AAX1117 | P006-138-01 | 68 | 6 |
| AAX1117 | P025-08-02 | 66 | 6 |

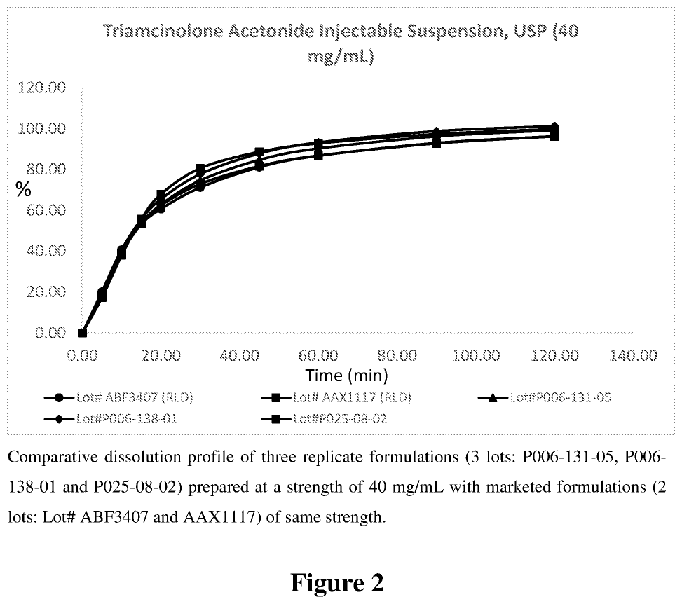

FIG. 2 is a comparison of the dissolution profiles of marketed products (2 lots) and test products of triamcinolone acetonide suspensions. FIG. 2 provides the comparative dissolution profile of three replicate formulations (3 lots: P006-131-05, P006-138-01 and P025-08-02) prepared at a strength of 40 mg/mL with marketed formulations (2 lots: Lot #ABF3407 and AAX1117) of the same strength.

From FIG. 2, it is clear that the dissolution profile of the marketed and test products are comparable because they have F₁ values <9 and F₂ values >60, as given in Table 1.

Triamcinolone acetonide injectable suspensions prepared according to the present invention method are stable at both room temperature and accelerated condition. Stability study results showed no significant change in particle size distribution, triamcinolone acetonide assay, preservative content and in impurities.

The Triamcinolone acetonide suspension injection prepared according to the present invention is capable of (i) controlling the particle size of triamcinolone acetonide in a suspension to obtain desired dissolution profile; ii) preventing triamcinolone degradation and other impurity formation, and iii) controlling viscosity of the suspension.

Without intending to be limited by theory, the inventors have developed the following theory as to how the process may protect multiple aspects of the triamcinolone acetonide, such as potency and particle size. Triamcinolone acetonide undergoes degradation by hydrolysis and oxidation when subjected to high temperatures in an aqueous medium. Water can act as an acid by formation of a hydronium ion (H₃O⁺) and catalyze the cleavage of triamcinolone acetonide. Further, if there is dissolved oxygen in the water, the triamcinolone acetonide can be oxidized. The current process may reduce the degradation of triamcinolone acetonide by using a combination of one or more of: (a) limiting the slurry volume relative to the final product weight, e.g., keeping the slurry at between 30-40% w/w of the final product weight; (b) removing any dissolved oxygen from the water; (c) using a nitrogen atmosphere when applying moist heat sterilization; and (d) limiting the sterilization exposure to 15 minutes at 122° C.

During exposure of triamcinolone acetonide to moist heat some of the molecules may be solubilized at the high temperature and then during the cooling phase some of the molecules may form larger particles due to the combined effect of aggregation and Ostwald ripening phenomenon. The addition of benzyl alcohol, carboxymethylcellulose sodium and sodium chloride to the active ingredient slurry prevents the degradation of triamcinolone acetonide and results in particles size increase of triamcinolone acetonide during the moist heat sterilization.

It is to be understood that the above description is intended to be illustrative and not restrictive. Many embodiments will be apparent to those of skill in the art upon reading the above description. The scope of the invention should, therefore, be determined with reference to the appended claims, along with the full scope of equivalents to which such claims are entitled. The disclosures of all articles and references, including patents, patent applications and publications, are incorporated herein by reference in their entirety and for all purposes.

### Example: Triamcinolone Acetonide Injectable Suspension Composition

| | | Quantity in |
|---|---|---|
| Sr. No | Ingredient | Percentage |
| 1 | Triamcinolone acetonide | 4.00 |
| 2 | Sodium chloride | 0.66 |
| 3 | Benzyl alcohol | 0.99 |
| 4 | Carboxymethylcellulose sodium | 0.63 |
| 5 | Polysorbate 80 | 0.04 |
| 6 | WFI | q.s. to 1 mL |

### Manufacturing Process:

The manufacturing process has the following steps:

### Step I.

a) In Tank 1, dissolve benzyl alcohol followed by sodium chloride in water for injection maintained at 55-60° C. under stirring.
b) Add carboxymethylcellulose sodium slowly to prevent formation of lumps.
c) Mix under stirring for 6 hours.
d) Allow the solution to come to room temperature.
e) Transfer the above solution to Tank 2 through an 8 μm filter.

### Step II.

a) In Tank 2, add the required quantity of Triamcinolone acetonide under stirring to form a slurry.
b) Pass the slurry through an inline homogenizer connected to the tank to break up any formed agglomerates. Keep the slurry under mixing throughout the compounding process.
c) Sparge with nitrogen to reduce dissolved and headspace oxygen level below 2 ppm.
d) Sterilize at 122° Celsius (15 to 90 minutes) under nitrogen atmosphere.

### Step III.

a) In Tank 3, dissolve polysorbate 80 in water for injection.
b) Filter the polysorbate solution through a 0.22 μm sterile filter into Tank 2 using aseptic quick connect.
c) Rinse with additional water for injection sufficient to make final batch weight and filter through a sterile filter into Tank 2.

### Step IV.

a) Transfer the product to a sterile holding tank (Tank 4).
b) Perform aseptic filling at 1 mL, 5 mL and 10 mL fill volume as per the requirement.

The sterile suspension of triamcinolone acetonide is prepared as described in the above Example. Analytical results of the formulation are summarized in Table 2.

TABLE 2

| | | | Triamcinolone | | Benzyl Alcohol | | Total | |
|---|---|---|---|---|---|---|---|---|
| | pH | | Acetonide Assay (%) | | Assay (%) | | Impurity (%) | |
| Time | 25° C./ | 40° C./ | 25° C./ | 40° C./ | 25° C./ | 40° C./ | 25° C./ | 40° C./ |
| (Months) | 60% RH | 75% RH | 60% RH | 75% RH | 60% RH | 75% RH | 60% RH | 75% RH |
| 0 | 6.33 | 6.33 | 109.0 | 109.0 | 105.9 | 105.9 | 0.112 | 0.112 |
| 1 | 6.45 | 6.34 | 102.9 | 102.5 | 100.0 | 99.70 | 0.108 | 0.125 |
| 2 | 6.43 | 6.37 | 100.9 | 100.9 | 100.7 | 100.4 | 0.113 | 0.108 |
| 6 | — | — | — | 102.4 | — | 101.6 | — | 0.205 |

The particle size of triamcinolone acetonide in the suspension prepared according to the present invention compared with the reference product and the particle size distribution (PSD) results are summarized in Table 3.

TABLE 3

| PSD Range | | Test Product |
|---|---|---|
| (μm) | Reference Product | (Present Invention) |
| D₁₀ | 1.90-2.45 | 1.84-3.25 |
| D₅₀ | 6.07-8.85 | 5.29-6.77 |
| D₉₀ | 13.80-29.20 | 10.9-13.30 |

It was observed from the above results that the triamcinolone acetonide suspension prepared in accordance with the present invention exhibited desirable properties in both the particle size of triamcinolone acetonide in suspension to obtain desired dissolution profile and having an impurity profile within the specified limit.

1. A sterile aqueous slurry of a water-insoluble steroid, benzyl alcohol, sodium chloride and sodium carboxymethylcellulose, wherein the potency of the steroid in suspension in particle form in the slurry is not reduced by application of moist heat sterilization to the slurry.

2. The sterile injectable suspension of claim 1, wherein the water-insoluble steroid is triamcinolone acetonide.

3. The sterile aqueous slurry of claim 2, wherein the triamcinolone acetonide is present at an amount of about 1% to about 10% by weight.

4. The sterile aqueous slurry of claim 2, wherein the concentration of triamcinolone acetonide in the slurry is between about 1% to about 20% la weight.

5. The sterile aqueous slurry of claim 2, wherein the slurry consists essentially of water, triamcinolone acetonide, benzyl alcohol, sodium chloride and sodium carboxymethylcellulose, whereby the potency of the triamcinolone acetonide is not reduced by application of moist heat sterilization to the slurry.

6. The sterile aqueous slurry of claim 2, wherein the slurry consists of water, triamcinolone acetonide, benzyl alcohol, sodium chloride and sodium carboxymethylcellulose.

7. The sterile aqueous slurry of claim 2, wherein the application of moist heat sterilization to the slurry increases the particle size of the triamcinolone acetonide in the slurry.

8. A sterile injectable suspension comprising the aqueous slurry of claim 1 and one or more pharmaceutically acceptable excipients selected from the group consisting of vehicle, suspending agent, surfactant, buffering agent, tonicity agent and preservative.

9. The sterile injectable suspension of claim 8, wherein the suspension comprises about 1.96% to about 19.6% of triamcinolone acetonide, about 1.65% to about 1.94% of benzyl alcohol, about 1.1% to about 1.29% of sodium chloride, about 1.05% to about 1.23% of sodium carboxymethylcellulose and one or more other pharmaceutically acceptable excipients.

10. The sterile injectable suspension of claim 8, wherein the suspension is not sterilized in a sterilization process that includes a step of a terminal sterilization.

11. The sterile injectable suspension of claim 8, wherein the particle size of triamcinolone acetonide in the suspension is from about 0.5 μm to about 30 μm.

12. The sterile injectable suspension of claim 7, wherein the viscosity of the suspension is between about 8 centipoise to about 50 centipoise and pH of between about 4.0 to about 8.

13. A process for preparing a sterile injectable suspension of a water-insoluble steroid, the process comprising the steps of:
a) preparing an aqueous slurry of a water-insoluble steroid, benzyl alcohol, sodium chloride and sodium carboxymethylcellulose; and
b) sterilizing the aqueous slurry by moist heat sterilization to form a sterile slurry, wherein the potency of the steroid in the suspension is not reduced by application of moist heat sterilization to the slurry.

14. The process of claim 13, further comprising:
c) forming a solution of a mixture of one or more of a vehicle and a surfactant;
d) filtering the solution of step c) through a sterile filter; and
e) combining the filtered solution of step d) with the sterile slurry of step b) to form a suspension.

15. The process of claim 13, wherein the water-insoluble steroid is triamcinolone acetonide.

16. The process of claim 13, wherein the water-insoluble steroid is triamcinolone acetonide and the concentration of triamcinolone acetonide in the slurry is between about 1% to about 20%.

17. The process of claim 13, wherein the sterilization of the aqueous slurry is performed under homogenization and/or a nitrogen atmosphere.

18. The process of claim 13, wherein the moist heat sterilization comprises heating the aqueous slurry to a temperature of about 115° C. to about 128° C. for about 15 to about 90 minutes.

19. The process of claim 13, wherein the particle size of triamcinolone acetonide in the suspension ranges from about 0.1 μm to about 30 μm.

20. The process of claim 14, wherein the process does not include a step of terminally sterilizing the suspension.
